(19) United States
(12) Patent Application Publication
(10) Pub. No.: US 2010/0168058 A1
(43) Pub. Date: Jul. 1, 2010

(54) NOVEL ANTAGONISTS OF THE TOLL-LIKE RECEPTOR 4

(76) Inventor: Leonardus Antonius B. JOOSTEN, LM BEUNINGEN (NL)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/443,681

(22) PCT Filed: Oct. 2, 2006
(86) PCT No.: PCT/EP2006/009528

§ 371 Date: Nov. 11, 2009

(51) Int. Cl. A61K 31/715; C07H 3/00; A61P 19/02

(52) U.S. Cl. 514/54; 536/123.1

(57) ABSTRACT

The present invention relates to a novel antagonist of the Toll-like receptor 4 (TLR-4). More specifically, the present invention relates to a lipopolysaccharide (LPS) isolated from the bacterium Bartonella quintana as a novel antagonist of the Toll-like receptor 4 (TLR-4). Further, the present invention relates to the use of said natural antagonist for the treatment of an autoimmune or inflammatory disease in a mammal and specifically for the treatment of rheumatoid arthritis (RA). Furthermore, the present invention relates to pharmaceutical compositions comprising a lipopolysaccharide (LPS) isolated from the bacterium Bartonella quintana.

The present invention relates to novel antagonists of the Toll-like receptor 4 (TLR-4). More specifically, the present invention relates to a lipopolysaccharide (LPS)isolated from the bacterium Bartonella quintana as a novel antagonist of the Toll-like receptor 4 (TLR-4). Further, the present invention relates to the use of said natural antagonist for the treatment of an autoimmune or inflammatory disease in a mammal such as a human, and specifically for the treatment of rheumatoid arthritis (RA).

The bacterium Bartonella quintana (B. quintana) is a pathogen initially described during World War I as the causative agent of trench fever, a disease associated with recurrent fever and headaches. In the past decades, Bartonella quintana infection has been identified in homeless people.

While most individuals with Bartonella quintana infection recover, some 5 to 10% will eventually develop chronic bacteremia, and subsequent complications such as chronic endocarditis in the absence of pre-existing heart-valve lesions.

New manifestations of Bartonella quintana infections such as bacillary angiomatosis, bacillary peliosis hepatis, and chronic lymphadenopathy have also been described, and these manifestations have been attributed to proliferative and anti-apoptotic effects of Bartonella quintana.

The bacterium Bartonella quintana was identified in the early literature as belonging to the genus Rochalimaea and was designated Rochalimaea quintana. However also other designations were used such as Rochalimaea weigli, Rochalimaea volhynica, Rochalimaea pediculi and Rochalimaea rocha-limae.

Based on revised DNA hybridization results and other phylogenetic, genotypic and phenotypic studies, the bacterium was reclassified as belonging to the genus Bartonella also comprising the members Bartonella bacilliformis, Bartonella vinsonii, Bartonella henselae and Bartonella elizabethae.

Presently, the genus Bartonella is characterized as gram-negative, oxidase negative, fastidious, aerobic, rod-shaped bacteria which best grow on blood enriched media in an atmosphere containing 5% carbon dioxide.

Like other gram-negative bacterial species such as Escherichia coli, the bacterium Bartonella quintana comprises an outer cell membrane in which lipopolysaccharide complexes, also designated as lipopolysaccharide or LPS, are associated.

In general, a lipopolysaccharide comprises a lipid portion, also designated as lipid A, and a polysaccharide portion. Although the Lipid A portion is generally conserved across species of bacteria, the Lipid A of some bacterial species is considerably more toxic than that of others.

Further, the polysaccharide portion of lipopolysaccharide in general contains a core and an “O-specific chain”. Minor variations in the structure of the O-polysaccharide provide enormous differences to the virulence of bacterial infections, i.e., the capacity of the bacteria to multiply and to infect.

In all living species, the first line of defense against microbial aggressions is constituted by innate immunity. During evolution, the innate immunity appears in invertebrates and plants, long before adaptive immunity, which appears in vertebrate.

Adaptive immunity induces acquired resistance against microorganisms through random somatic rearrangements of genes encoding immunoglobulins and T cell receptors, thus generating a high level of diversity of receptors in response to microbial aggressions. Acquired resistance is not vertically transmitted in the germ line and reflects the “infectious history” of every individual.

In contrast, innate immunity relies on recognition of antigens by a small number of specific receptors designated as Pattern-Recognition Receptors (PRR) and is vertically, through the germ line, transmitted by germinal cells.

The Pattern-Recognition Receptors(PRR) are expressed on macrophages, dendritic cells and B lymphocytes and recognize highly conserved antigenic structures, termed Pathogen-Associated Molecular Patterns (PAMP), such as lipopolysaccharides, peptidoglycanes and lipoteichoic acids.

The Pattern-Recognition Receptors (PRR) are secreted or expressed at the cell surface of cells to induce endocytosis or signaling through the Toll-like receptors or TLRs.

The recognition of antigens by these Toll-like receptors (TLRs) induces an immediate inflammatory response and triggers adaptive immunity. Toll-like receptors (TLRs) induce an inflammatory response against microorganisms through, for example, NF-kappaB, a cytoplasmic factor controlling transcription of many genes, including cytokines (TNF, INF, IL-1, IL-2, IL-8, IL-12) and defensines.

The Toll-like receptor (TLR) bacterial antigen lipopolysaccharide (LPS) is recognized in mammals by a receptor complex composed of CD14, Toll-like receptor 4 (TLR-4), and MD-2. The detailed mechanisms of how TLR-4 transmits the signal induced by the lipopolysaccharide (LPS) over the outer cell membrane of mammalian cells remains to be elucidated.

However, in agreement with other types of plasma membrane receptors, aggregation of Toll-like receptor 4 (TLR-4) by immobilized TLR-4 antibodies was sufficient to induce signaling, suggesting a mechanism of action through multimerization.

Pharmacological disruption of the Golgi apparatus did not inhibit LPS induced NF-kappaB activation. Further, lipopolysaccharide (LPS) stimulation recruited the adapter molecule MyD88 to the inside of the plasma membrane. Hence, these data indicate that lipopolysaccharide (LPS) signaling through the Toll-like 4 receptor appears to commence on the plasma membrane and is independent of trafficking to the Golgi.

Considering the role of Toll-like receptors as a critical link between the innate (germ line transmitted) immune response and the adaptive (acquired) immune response, the idea has emerged that a continuous activation and/or dysregulation of Toll-like receptor signaling might contribute to the pathogenesis of autoimmune diseases such as rheumatoid arthritis (RA), Crohn's disease, arteroscelorose, type I diabetes mellitus, Wegener's granulomatosis, multiple sclerosis, ulcerative colitis, etc.

Indeed, Toll-like receptor ligands of exogenous origin such as bacterial peptidoglycans and CpG-containing DNA, activating TLR2 and TLR9 respectively, have been found in sinovial fluids of patients with autoimmune diseases such as rheumatoid arthritis (RA).

In experimental animal models, Toll-like receptor ligands have repeatedly been used to induce autoimmune diseases in susceptible animals. For instance, intra-articular injection of streptococcal cell wall fragments, dsRNA or CpG-containing DNA, which mainly signal through Toll-like receptor 2, Toll-like receptor 3 and Toll-like receptor 9, respectively, can induce arthritis.

Furthermore, lipopolysaccharide (LPS) from the outer membrane of Gram-negative bacteria, signaling through Toll-like receptor 4 (TLR-4), has extensively been used to aggravate or reactivate arthritis in distinct animal models. In addition, lipopolysaccharide (LPS) has been demonstrated to circumvent the interleukin (IL)-1 dependence of the serum-transferred arthritis model K/B×N.

In spite of these properties of a variety of Toll-like receptor ligands, the role of Toll-like receptor signaling in autoimmune diseases is presently not clear. Alternatively, Toll-like receptors can also be activated by several endogenous ligands, which are released from stressed or damaged host tissues.

In this respect, Toll-like receptor 4 (TLR-4) can recognize extracellular matrix components such as heparan sulphate and extra domain A (EDA) of fibronectin, and Toll-like receptor 2 (TLR-2) and Toll-like receptor 4 (TLR-4) can both recognize the matrix component biglycan. Toll-like receptor (TLR) activation by these self-antigens can potentially promote the development of autoimmune diseases.

A critical role of Toll-like receptors (TLRs) in autoimmunity is supported by the finding that autoreactive B cells can be activated by RNA-associated as well as DNA-associated autoantigens via sequential engagement of the B cell receptor and Toll-like receptor 7 (TLR-7) or Toll-like receptor 9 (TLR-9), respectively. These findings may have implications for the understanding of the pathogenesis of systemic lupus erythematosus.

Some of the endogenous Toll-like receptor (TLR) ligands can be found in arthritic joints. It has recently been shown that RNA released from necrotic synovial fluid cells of rheumatoid arthritis (RA) patients can activate Toll-like receptor 3 (TLR-3) on rheumatoid arthritis (RA) synovial fibroblasts.

In addition, serum and synovial fluid of rheumatoid arthritis patients can activate a Toll-like receptor 4 (TLR-4) expressing Chinese Hamster Ovary (CHO) cell line, suggesting the presence of Toll-like receptor 4 (TLR-4) ligands in rheumatoid arthritis joints.

Further evidence for a potential role of Toll-like receptor 4 (TLR-4) has come to the fore from a recent report demonstrating a decreased susceptibility to rheumatoid arthritis in individuals with the Toll-like receptor 4 (TLR-4) functional variant Asp299Gly.

Hence, given the suspected role of Toll-like receptors (TLRs) in the development and/or maintenance of autoimmune diseases and their known role in infectious diseases such as viral, bacterial and fungal infections, there is a need in the art to identify and develop antagonists, i.e., substances that tend to nullify the action of another substance, as a drug that binds to a cell receptor without eliciting a biological response, of Toll-like receptors (TLRs) such as the Toll-like 4 receptor (TLR-4).

Therefore, one of the objects of the present invention is to provide such antagonists of the Toll-like receptors (TLRs) and specifically the Toll-like receptor 4 (TLR-4).

According to the present invention, this object, amongst others, was met by the surprising discovery that a natural occurring compound, i.e., the lipopolysaccharide (LPS) from Bartonella quintana, was not only able to interact with the Toll-like receptor 4 (TLR-4), but, in addition, was also able to inhibit or prevent the signal transduction through this receptor, thereby counteracting the biologic effects caused by the direct or indirect activation of this receptor.

Therefore, according to one aspect, the present invention relates to the use of Bartonella quintana lipopolysaccharide (LPS) for the preparation of a medicament for the treatment of an autoimmune, an inflammatory disease, and/or an infection, preferably such as infections accompanied by overwhelming inflammation symptoms, for example during SIRS and/or sepsis, in a mammal.

According to a preferred embodiment of the present invention, the mammal to be treated using the compound according to the present invention is a human mammal suffering from an autoimmune disease or an infection.

Although the diseases to be treated using the compound according to the present invention can be caused by activation of different signal transduction pathways wherein the Toll-like receptor (TLR) signal transduction pathway is indirectly involved, according to a preferred embodiment of the present invention, the diseases to be treated are a direct result of activation of the Toll-like receptor (TLR) pathway.

Therefore, according to another aspect, the present invention preferably relates to the above described use, wherein the treatment comprises inhibition of the Toll-like receptor (TLR) signaling, most preferably inhibition of the Toll-like receptor 4 (TLR-4) signaling, although it is considered to be within the scope of the present invention that the compound according to the present invention is also able to exert its beneficial effect in the treatment of autoimmune diseases wherein the activation of the Toll-like 4 receptor (TLR-4) is not the primary cause, i.e., indirectly involved.

The term “directly” involved in the autoimmune disease as used herein is meant to indicate that there is a direct relation between the activation of the receptor and the occurrence of the disease. In other words, activation of the receptor is significantly associated with the disease.

The term “indirectly” involved in the autoimmune disease as used herein is meant to indicate that there is an indirect relation between the activation of the receptor and the occurrence of the disease.

Although the activation of the receptor does not always significantly correlate with the occurrence of the disease, the Toll-like receptor (TLR) signaling transduction route can play a roll in, for example, the severity of the disease, the occurrence of side effects or complications, or the development of the disease in a mammal, such as a human mammal.

The above indirect involvement of the Toll-like receptor (TLR) transduction pathway and especially the Toll-like receptor 4 (TLR-4) transduction pathway in an autoimmune disease or infection to be treated is also present in the Toll-like receptor (TLR) transduction pathway itself.

For example, although Toll-like receptor 9 (TLR-9) can be directly activated as a consequence of the disease, inhibiting, for example, the Toll-like receptor 4 (TLR-4) can influence other components of the Toll-like receptor (TLR) signal transduction pathway thereby decreasing the biologic effect caused by the direct activation of Toll-like receptor 9 (TLR-9).

In other words, because of the complex interactions and feed-back mechanisms between signal transduction pathways, gene expression, hormonal and neurological responses, the humoral and cellular immune system, metabolites and breakdown products, etc, and the complex interactions and feed-back mechanisms within a specific signal transduction pathway itself such as the Toll-like receptor (TLR) signal transduction pathway, the present invention also relates to the treatment of autoimmune diseases and infections wherein the activation of the Toll-like receptor 4 (TLR-4) is not directly involved, however inhibition of this receptor is beneficial for the treatment of the autoimmune disease or the infection.

An example of the above “indirect” scenario is an autoimmune disease or an viral infection causing cellular lysis and the subsequent release of extracellular matrix components such as heparan sulphate and extra domain A (EDA) of fibronectin.

Although the autoimmune disease or the virus, inherently lacking lipopolysaccharide (LPS), are not able to directly activate the Toll-like receptor 4, because of the subsequent cellular lysis, i.e., a secondary or indirect effect of the autoimmune disease or viral infection, the binding of the extracellular matrix components to the receptor can potentially lead to activation of the receptor thereby triggering undesirable secondary biological responses.

Preferred autoimmune or inflammatory diseases according to the present invention are rheumatoid arthritis, Crohn's disease, bacterial, viral and fungal infections, atherosclerosis, type I diabetes mellitus, Wegener's granulomatosis and multiple sclerosis and inflammatory bowel disease. Most preferred is rheumatoid arthritis.

Although the present invention is preferably related to the lipopolysaccharide (LPS) of Bartonella quintana, structurally and functionally similar lipopolysaccharide (LPS) fragments thereof are also contemplated to fall within the scope of the present invention.

Without wishing to be bound to any theory, in line with a low endotoxic potency of lipopolysaccharide (LPS) from Bartonella quintana, it was recently reported that LPS isolated from another family member, Bartonella henselae, is a penta-acylated deep-rough lipopolysaccharide (LPS) with endotoxic activities at least 1000-fold less potent than those of Salmonella enterica.

It has also been reported that Bartonella quintana lipopolysaccharide (LPS) has a similar deep-rough structure.

However, differences with Bartonella henselae lipopolysaccharide (LPS) must also be present, as Bartonella quintana lipopolysaccharide (LPS) is completely unable to stimulate cytokines at concentrations as high as 10 mg/ml. However, to nature of these observed differences remains to be established.

The above indicates that the antagonist properties of the preferred Bartonella quintana lipopolysaccharide (LPS) potentially could also be obtained using a fragments of the preferred compound according to the present invention.

Hence, the present invention also relates to functionally similar or equivalent fragments of the Bartonella quintana lipopolysaccharide (LPS) according to the present invention.

Since the compounds according to the present invention are beneficial for the treatment of autoimmune diseases and inflammatory diseases, the present invention also relates to Bartonella quintana lipopolysaccharide (LPS) for use as a medicament and a method for treating an autoimmune or inflammatory disease comprising administering a therapeutic amount of Bartonella quintana lipopolysaccharide to a mammal suffering from an autoimmune or inflammatory disease.

All described uses and methods according to the present invention are preferably carried out by using a medicament comprising Bartonella quintana lipopolysaccharide and one or more pharmaceutically acceptable excipients and carriers, well known and generally used in the pharmaceutical art.

Such medicament can, for example, be formulated for oral, parenteral, or implantable administration, etc., using methods well known to the skilled person.

The formulated medicament can take the form, for example, of an injectable solution or suspension, a particulate or microparticulate, a tablet, a capsule, a suppository, an immediate release formulation, a sustained release formulation, an implantable pad, a cream, a lotion, etc.

Another preferred aspect of the present invention relates to the use of a Toll-like 4 receptor (TLR-4) antagonist for the preparation of a medicament for the treatment of rheumatoid arthritis.

According to preferred embodiments of this aspect, the Toll-like 4 receptor (TLR-4) antagonist is a lipopolysaccharide (LPS), or a functional equivalent thereof, of Bartonella quintana.

The present invention shall now be below illustrated using preferred detailed examples. The examples are given to demonstrate the present invention, however, are not intended to limit the scope of protection of the present invention which is solely determined by the appended claims.

In the examples, demonstrating preferred embodiments of the present invention, reference will be made to the appended drawings. In the drawings, the following is shown for illustrative purposes:

### DRAWINGS

FIG. 1: FIG. 1a: human PBMC were stimulated for 24 hours with 1×10⁶ heat killed B. quintana microorganisms/mL, in the presence of either control IgG1 (open bars) or an anti-TLR-4 antibody (10 mg/mL, hatched bars). Unstimulated cells are presented as solid bars.

FIG. 1b: human PBMC were stimulated for 24 hours with E. coli LPS (10 ng/mL, open bars) or B. quintana LPS (1 mg/mL, hatched bars).

Unstimulated cells are presented as solid bars. Cytokines were measured by specific ELISA. Data are presented as means±S.D. (n=6).

FIG. 2: FIG. 2a: human PBMC were stimulated (+) or not (−) for 24 hours with E. coli LPS at a concentration of 10 ng/mL. Before stimulation with E. coli LPS, the cells were preincubated with either RPMI or with B. quintana LPS, in concentrations ranging from 1 to 100 ng/mL. TNF was measured by specific ELISA.

FIG. 2b: PBMCs were preincubated with B. quintana LPS, 100 ng/ml, and thereafter stimulated with E. coli LPS, 10 ng/mL. mRNA of proinflammatory cytokines was measured thereafter.

FIG. 2c: CHO cells were transfected with either hCD14 or with a combination of hTLR4 and hCD14 and stimulated with E. coli LPS (1 mg/mL), B. quintana LPS (10 mg/mL), or a combination of both. Expression of CD25 on the cell membrane was measured by FACS analysis. The TLR2 agonist Pam3Cys (5 mg/mL) served as negative control stimulus. Data are presented as means±S.D. (n=6, *p<0.05).

FIG. 3: FIGS. 3a and 3b: PBMCs were stimulated with 10 ng/ml E. coli LPS after preincubation with 0, 1, 10, 100, 1000 and 10,000 ng/ml of B. quintana LPS. Ratios of B. quintana LPS (TLR-4 antagonist) to E. coli LPS concentrations are depicted on the X-axis. Data are expressed as the mean of 4 donors and are representative for 3-independent experiments.

FIG. 3c: Macrophages were incubated with 10 ng/ml E. coli LPS, 10 mg/ml EDA and 10 mg/ml HS or left untreated after they were pretreated with 10× higher concentrations of B. quintana LPS. EDA and HS were mixed with 10 mg/ml polymyxin B prior to use. Data are expressed as mean±SEM. EDA, extra domain A of fibronectin; HS, heparan sulphate; N.d., not detected.

FIG. 4: FIG. 4a: Mouse macrophages were stimulated with 10 ng/ml E. coli LPS or 1 mg/ml B. quintana LPS and cytokines were measured by Luminex assay.

FIGS. 4b and 4c: Immature monocyte-derived DCs were incubated with 2 mg/ml E. coli LPS or 1 mg/ml ssPolyU for 48 hours after a preincubation with 10 times higher concentrations of B. quintana LPS. DC maturation was determined based on MHC class II and CD83 expression (FACS analysis).

FIG. 5: Mice received 3 i.p. injections of 400 mg/kg TLR-4 antagonist, 3 mg/kg TNFbp (Enbrel) or 0.2 ml saline once every two days started before the onset of arthritis.

FIG. 5a: Macroscopic arthritis score expressed as mean±SEM (scale 0-2 for each paw).

FIG. 5b: Histological analysis of the knee joints on day 6 of treatment expressed as mean±SEM of two independent experiments (scale 0-3). All parameters were scored by two blinded observers; n≧10 mice per group per experiment.

FIG. 5c: Representative histological images of TLR-4 inhibition. Tissue sections were stained using the H&E staining (upper panels) to study the inflammatory cell influx and chondrocyte death (open arrow) or using the Safranin O staining (lower panels) for PG-depletion and cartilage and bone destruction. PG-depletion is apparent from loss of staining. Original magnification ×200 for H&E and ×400 for Safranin O staining. B, bone; C, cartilage; F, femur; JS, joint space; P, patella. * P<0.05.

FIG. 6: FIGS. 6a to 6c: CIA mice were treated using 4 i.p. injections of 2 mg/kg TLR-4 antagonist, 3 mg/kg TNFbp (Enbrel) or 0.2 ml saline after a macroscopic inflammation score of 0.5-1 was reached on a scale up to 8 per mouse.

FIG. 6a shows macroscopic arthritis score expressed as mean±SEM (scale 0-2 for each paw).

FIG. 6b shows histological analysis of the knee joints on day 4 of treatment presented as mean±SEM (scale 0-3).

All parameters were scored by two blinded observers; n=10 mice per group.

FIG. 6c shows histological images of the knee joints. Cell influx and chondrocyte death (open arrow) were scored on H&E-stained and cartilage and bone damage (stealth arrows) were scored on Safranin O-stained tissue sections. Original magnification ×200 for H&E and ×400 for Safranin O staining.

FIG. 6d: Macroscopic arthritis score in IL-1Ra−/− mice treated with TLR-4 antagonist (400 mg/kg), TNFbp (3 mg/kg) or saline or BSA as controls three times a week after the arthritis onset.

FIGS. 6e and 6f: Incidence (shown in FIG. 6e) and severity (shown in FIG. 6e) of arthritis in IL-1Ra^{-/-} TLR-4 KO as compared to IL-1Ra^{-/-} TLR-4 WT mice. BSA, bovine serum albumin; IL-Ra, IL-1 receptor antagonist; WT, wild type; KO, knockout. * P<0.05.

FIG. 7: FIG. 7a: Immunohistochemical staining of IL-1b in the knee joint. Arrows show high IL-1 expression in synovium at the site of bone erosion. Original magnification ×400. B, bone; C, cartilage; S, synovium.

FIGS. 7b and 7b: Prophylactic (FIG. 7b) and therapeutic (FIG. 7c) treatments with TLR-4 antagonist resulted in lower expression of IL-1b protein in articular chondrocytes and synovial tissue.

FIGS. 7d and 7e: Serum levels of anti-type II collagen antibodies were not affected by prophylactic (FIG. 7d) or therapeutic (FIG. 7e) treatment with TLR-4 antagonist (ELISA). * P<0.05, ** P<0.01 and *** P<0.001

FIG. 8: Mice received 0.2 ml saline, 10 mg E. coli LPS or 50 mg B. quintana LPS systemically. Serum was isolated after 90 minutes and 4 hours.

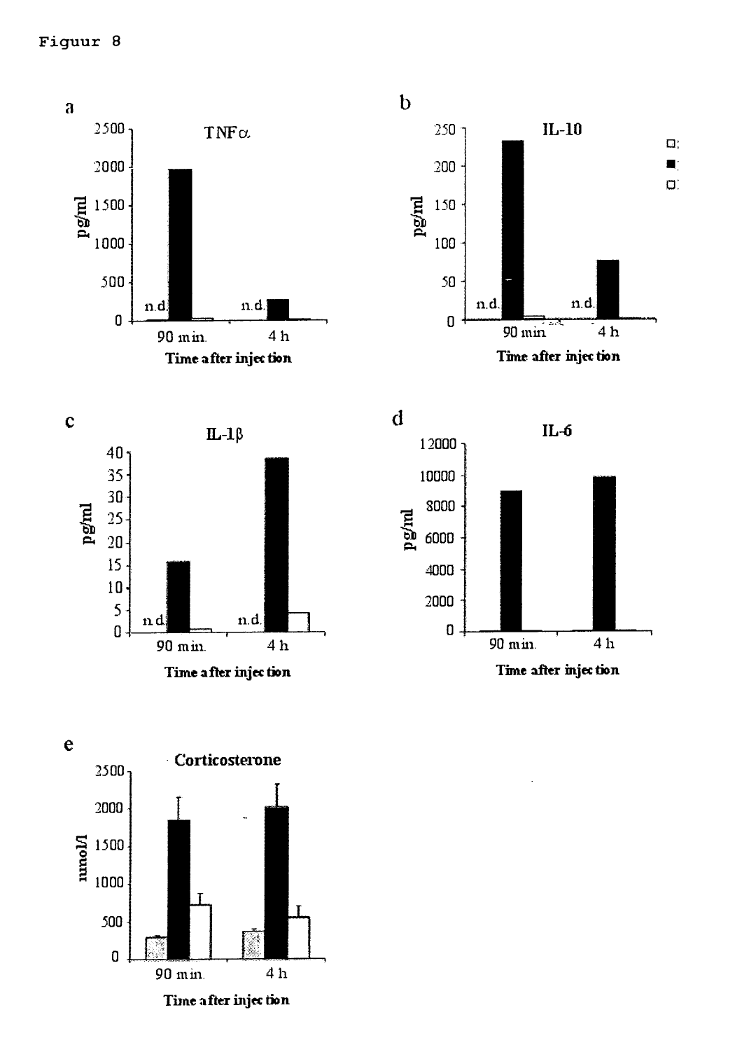

Concentration of TNFa (FIG. 8a), IL-10 (FIG. 8b), IL-1b (FIG. 8c) and IL-6 (FIG. 8d) were measured using the Luminex technique. Corticosterone levels (FIG. 8e) were determined using radio-immunoassay. Data represent means of at least 6 mice per group. N.d., not detected.

### EXAMPLES

### Example 1

### Bartonella quintana Lipopolysaccharide is a Natural Antagonist of Toll-Like Receptor 4 (TLR-4)

### Introduction

In the present example, the biologic activities of B. quintana LPS in terms of induction of proinflammatory cytokines and of interaction with Toll-like receptors (TLRs) and other species of lipopolysaccharide (LPS) was investigated.

### Materials and Methods

### Reagents and Microorganisms

LPS (E. coli serotype 055:B5) was purchased from Sigma Chemical Co., and synthetic Pam3Cys was purchased from EMC Microcollections (Tübingen, Germany).

The B. quintana Oklahoma strain was kindly provided by Prof. D. Raoult (Marseille, France) and grown on sheep blood agar at 37° C. in a 5% CO₂ atmosphere. For the stimulation experiments, the five days cultures of B. quintana were heat-killed for 60 minutes at 56° C.

B. quintana LPS was extracted either by a single step phenol-water extraction as previously described by Liberto M. C. et al. “In vitro Bartonella quintana infection modulates the programmed cell death and inflammatory reaction of endothelial cells.”, Diagn. Microbiol. Infect. Dis., 2003, vol. 45: pp 107-115; and by a two-step extraction method described by Hirschfeld M. et al. “Inflammatory signaling by Borellia burgdorferi lipoproteins is mediated by Toll-like receptor 2.”, J. Immunol., 1999, vol. 163: pp 2382-2386, which eliminates contamination with proteins.

### Isolation of Peripheral Blood Mononuclear Cells (PBMC) and Stimulation of Cytokine Production

After informed consent, venous blood was drawn from the cubital vein six healthy volunteers into three 10 ml lithium-heparin tubes (Monoject, s-Hertogenbosch, The Netherlands).

The PBMC fraction was obtained by density centrifugation of blood diluted 1:1 in pyrogen-free saline over Ficoll-Paque (Pharmacia Biotech AB, Uppsala, Sweden).

Cells were washed twice in saline and suspended in culture medium (RPMI 1640 DM) supplemented with gentamicin 10 mg/ml, L-glutamine 10 mM and pyruvate 10 mM. The cells were counted in a Coulter counter (Beckman Coulter, Mijdrecht, The Netherlands) and the number was adjusted to 5×10⁶ cells/ml.

5×10⁵ PBMC in a 100 ml volume were added to round-bottom 96-wells plates (Greiner, Alphen a/d Rijn, The Netherlands) and incubated with either 100 ml of culture medium (negative control), or the various stimuli: B. quintana bacteria (1×10⁶ microorganisms/ml), B. quintana LPS (concentrations up to 10 mg/ml), E. coli LPS (10 ng/ml).

To determine the role of TLR-4 in the induction of cytokines by B. quintana LPS, PBMC were preincubated for 30 minutes with 10 mg/mL control IgG1 or a blocking anti-TLR-4 antibody (eBioscience, AMDS Malden, The Netherlands).

Similarly, the TLR-4 antagonistic properties of B. quintana LPS were assessed by preincubating the cells with various concentrations of B. quintana LPS, 30 minutes before stimulation with E. coli LPS.

### Cytokine Measurements

Human TNFalpha, IL-1b, IL-8 and IL-6 concentrations were measured by commercial ELISA kits (Pelikine Compact, CLB, Amsterdam, The Netherlands), according to the instructions of the manufacturer.

### RNA Extraction

Total RNA was extracted from 10×10⁶ cells using 1 ml TRIzol reagent (Sigma, St. Louis, Mo., USA). Subsequently, 200 μl chloroform and 500 μl 2-propanol (Merck, Darmstadt, Germany) were used to separate the RNA from DNA and proteins. Finally, after a wash step with 75% ethanol (Merck, Darmstadt, Germany), the dry RNA was dissolved in 30 μl of water.

### PCR Amplification

To obtain cDNA, 2 μg DNase treated total RNA was reverse transcribed with oligo dT primers (0.01 μg/ml) in a RT-PCR with a total volume of 20 μl.

Subsequently, quantitative PCR was performed using ABI/PRISM 7000 Sequence Detection System (Applied Biosystems, Foster City, Calif.). PCR's of GAPDH, IL-1β, TNF-α, IL-6 were performed with Sybr Green PCR Master Mix (Applied Biosystems, Foster City, Calif.), 5 μl 1/20 diluted cDNA and primers in a final concentration of 300 nmol/l in a total volume of 25 μl.

Primers were developed using primer express (Applied Biosystems, Foster City, Calif.): 5′-CCT CTG ATG GCA CCA CCAG-3′ for TNF-α, 5′-GGC AAG TCT CCT CAT TGA ATC C-3′ for IL-6, and 5′-TGG GTA ATT TTT GGG ATC TAC ACT CT-3′ for IL-1β.

Quantification of the PCR signals of each sample was performed by comparing the cycle threshold values (Cₜ) in duplicate, of the gene of interest with the Cₜ values of the GAPDH housekeeping gene. All primers were validated according to the protocol and the standard curves were all within the tolerable range.

### Signaling Through Human TLR-4 in a Transfected Cell Line

Chinese hamster ovary (CHO) fibroblasts stably transfected with human CD14 (3E10-CD14), a combination of CD14 and TLR-4 (3E10-TLR-4), were a kind gift from Dr. Robin Ingalls.

Cell lines express inducible membrane CD25 under control of a region from the human E-selectin (ELAM-1) promoter containing NF-kappaB binding sites. Cells were maintained at 37° C. and 5% CO2 in HAM's F12 medium (Gibcom, Invitrogen, Breda, the Netherlands) supplemented with 10% FCS, 0.01% L-glutamine, 50 mg/mL gentamicin and 400 U/mL hygromycin and 0.5 mg/mL of G418 (for 3E10-TLR2) or 0.05 mg/mL of puromycine (for 3E10-TLR-4) as additional selection antibiotics.

TLR-4 expression was confirmed by flow cytometry (Coulter Epics XL-MCL, Beckman Coulter, Mijdrecht, the Netherlands) using PE-labelled anti-TLR-4 antibody (clone HTA125) (Immunosource, Halle-Zoersel, Belgium).

For stimulation experiments, 500 ml of cells in culture medium at a density of 1×10⁵/mL were plated in 24-well culture plates. After an overnight incubation, cells were incubated with control medium, Pam3Cys (10 mg/ml), E. coli LPS (1 mg/ml), B. quintana LPS (10 mg/ml), or a combination of E. coli LPS and B. quintana LPS, for 20 hours at 37° C.

Thereafter, cells were harvested using trypsin/EDTA (Cambrex, East Rutherford, N.Y., USA) and prepared for flow cytometry (Coulter FACS-scan). CD25 expression of the CHO-cells was measured using FITC-labelled anti-CD25 (DAKO, Glostrup, Denmark), and expressed as folds-over-mean increase.

### Statistical Analysis

The human experiments were performed in triplicate with blood obtained from six volunteers. The differences between groups were analyzed by unpaired Student t-test, and where appropriate by paired t-test. The level of significance between groups was set at p<0.05. The data are given as means ±S.D.

### Results and Discussion

The first sets of experiments were designed to explore the induction of proinflammatory cytokines by whole B. quintana microorganisms, and the role of TLR-4 in this induction.

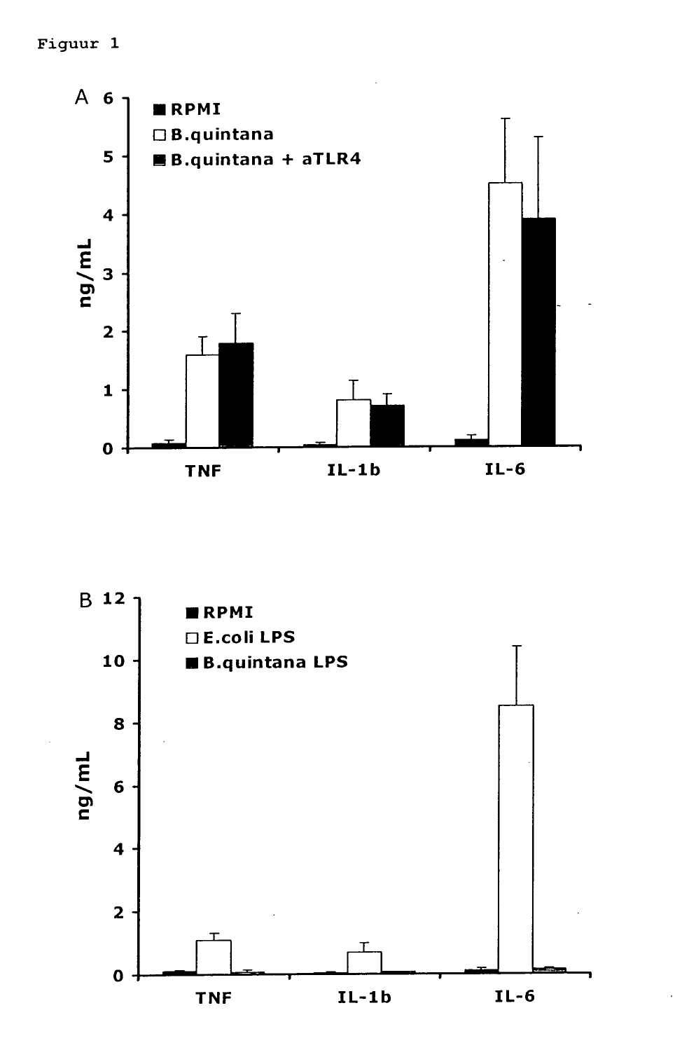

As shown in FIG. 1a, heat-killed B. quintana induced the production of proinflammatory cytokines by human PBMC. However, a blocking TLR-4 antibody did not influence the capacity of B. quintana to stimulate the production of these cytokines. This finding shows that the stimulation of cytokines by B. quintana is either not induced by its LPS component, or that B. quintana LPS stimulates cytokines through a TLR-4-independent mechanism. In line with this, stimulation of cytokines by the related microorganism B. henselae is mediated mainly by the proteinaceousVirB type IV secretion system, rather than LPS.

On the other hand, some LPS species, such as those isolated from non-enteric bacteria, Legionella pneumophila, Leptospira interrogans or Porphyromonas gingivalis interact with TLR-2 rather than with TLR-4, which might also explain the TLR-4-independence of B. quintana-induced cytokines.

This is in accordance with a recent study in which it is reported that cytokine stimulation by B. quintana is mediated almost entirely by TLR-2.

To investigate these two above-mentioned possibilities, B. quintana LPS was isolated in a two-step purification process which warrants the absence of protein contaminants. As shown in FIG. 1b, B. quintana LPS did not stimulate the production of TNFalpha, IL-1b and IL-6 in human PBMC. IL-8 was also not induced by B. quintana LPS (not shown). Similar negative results were obtained when peritoneal murine macrophages were stimulated (not shown).

These results demonstrate that B. quintana LPS is not able to induce cytokine production, and suggests that it is devoid of direct biological activity. This effect differs from a recent report in which B. quintana LPS has been reported to induce the release of the chemokine IL-8, while also being able to modulate the apoptosis of endothelial cells.

One crucial difference with these initial reports is the method of LPS purification. Indeed, when we stimulated PBMC with LPS only partly purified with the single-step method employed in the previous studies we also observed TLR-2 dependent stimulation of cytokines (not shown). However, the single step purification is prone to retain a high percentage of protein contaminants in the LPS preparation, which likely explains this discrepancy.

To investigate whether B. quintana LPS functions as TLR-4 antagonist, human PBMC were preincubated with various concentrations of B. quintana LPS, before stimulating the cells with E. coli LPS.

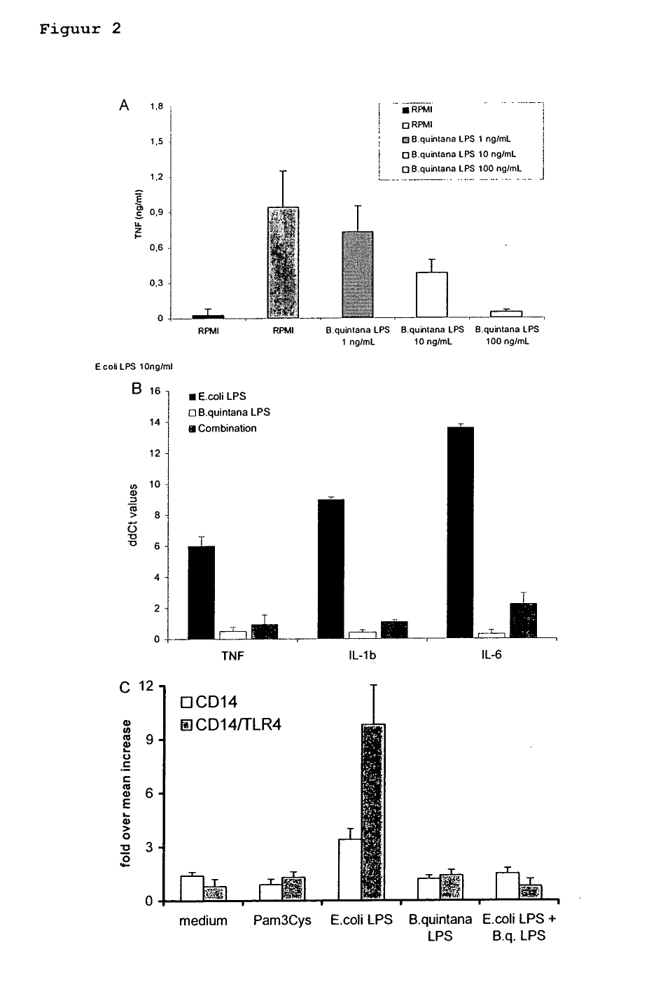

As shown in FIG. 2a, B. quintana LPS completely abolished E. coli LPS-induced TNF-α production when ratios of at least 10:1 were used, and inhibited the induction of cytokines by approximately 50% when 1:1 ratios between B. quintana LPS and E. coli LPS were employed.

In addition, when ratio 10:1 were used, B quintana LPS was able to completely inhibit also the expression of proinflammatory cytokines TNF-α, IL-1β and IL-6 mRNA from E. coli LPS challenged human PBMCs (FIG. 2b).

The inhibitory effect of B. quintana LPS was exerted by blocking TLR-4, as further demonstrated also by the blockade of E. coli LPS-induced stimulation of CHO cells transfected with TLR-4 (FIG. 2C). These results demonstrate that B. quintana LPS is a potent TLR-4 antagonist.

### Conclusion

The present example demonstrates that stimulation of cytokines by B. quintana is independent of both its LPS component and TLR-4. Moreover, while being unable to stimulate cytokine production, B. quintana LPS is a potent TLR-4 antagonist. Because TLR-4 proinflammatory signals are involved in a variety of pathologic inflammatory reactions, the use of the TLR-4 antagonistic properties of B. quintana LPS is of therapeutic value for both autoimmune and inflammatory diseases.

### Example 2

### Inhibition or Gene Deletion of TLR-4 Breaks the Inflammatory Loop in Chronic Autoimmune Arthritis

### Introduction

The present example demonstrates the involvement of TLR-4 activation in the development of chronic destructive arthritis using the collagen-induced arthritis (CIA) model and the spontaneous arthritis model in IL-1 receptor antagonist deficient (IL-1Ra^{-/-}) mice. CIA is an autoimmune model of arthritis based on auto-antibodies and T cell immunity against type II collagen (CII).

Here, it is demonstrated that inhibition of TLR-4 signaling in CIA mice using a naturally occurring TLR-4 antagonist substantially suppresses both clinical and histological characteristics of early-phase as well as established arthritis.

### Materials and Methods

### Animals

Male DBA-1/Bom mice were purchased from Bomholtgård (Ry, Denmark). IL-1Ra^{-/-} and TLR-4^{-/-} mice, both in BALB/c background; were kindly provided by Dr. M. Nicklin (Sheffield, England) and Prof. S. Akira (Osaka, Japan), respectively.

IL-1Ra^{-/-} (TLR-4^{+/+}) and TLR-4^{-/-} (IL-1Ra^{+/+}) mice were crossed and then offsprings heterozygous for both IL-1Ra and TLR-4 were intercrossed to obtain homozygous IL-1Ra^{-/-} mice that were either TLR-4^{+/+} or TLR-4^{-/-}. The mice were housed in filter-top cages, and water and food were provided ad libitum. Age- and gender-matched counterparts were used in experiments. Animal studies were approved by the Institutional Review Board and were performed according to the related codes of practice.

### Preparation and Purification of TLR-4 Antagonist

TLR-4 antagonist was LPS derived from the cell membrane of the Gram-negative bacterium Bartonella quintana. B. quintana Oklahama strain was kindly provided by Prof. D. Raoult (Marseille, France) and cultured on sheep blood agar at 37° C. with 5% CO₂. The 5 days cultures of B. quintana were heat-killed at 56° C. for 60 minutes and LPS was extracted using a two-step phenol-water extraction method as described previously to remove proteins and lipids (Manthey C. L. and Vogel S. N. “Elimination of trace endotoxin protein from rough chemotype LPS.”, Journal of Endotoxin Research, 1994, vol. 1, pp 84.

### Isolation, Culture and Stimulation of Human PBMC and Murine Resident Peritoneal Macrophages

PBMCs were isolated from heparanized venous blood by density gradient centrifugation over Ficoll-hypaque and cultured. Murine macrophages were isolated from naive DBA/1 mice by the lavage of the peritoneal cavity using 10 ml cold medium (DMEM+10% FCS). Adherent cells were harvested and cultured for four days before use.

For cytokine production, hPBMCs and murine macrophages were preincubated with TLR-4 antagonist for 30 minutes, then exposed to purified E. coli LPS (10 ng/ml), Pam3Cys (10 mg/ml), Poly I:C (25 mg/ml), IL-1 (10 ng/ml), TNFalpha (10 ng/ml), EDA of fibronectin (1 mM) and Heparan sulphate (HS, 10 mg/ml) for 24 hours.

EDA and HS were incubated with 10 mg/ml of Polymyxin B for 30 minutes prior to use in order to disable the possible LPS contamination. Concentration of TLR-4 antagonist was 10 times higher than these stimuli unless mentioned otherwise.

### Measurement of Cytokines

Cytokine concentrations (except TGFbeta) in culture supernatants and mice sera were determined using the Bioplex cytokine assays from Bio-Rad. TGFbeta concentrations were measured using ELISA (R&D systems) following the manufacturer's instruction.

### Generation and Maturation of Monocyte-Derived DCs

Immature DCs (iDCs) were generated from adherent monocytes. For DC maturation, 1.10⁶ iDCs were incubated with 2 mg/ml purified E. coli LPS or 1 mg/ml TLR7 ligand ssPolyU for 48 hours after a Preincubation with 10 times higher concentrations of TLR-4 antagonist.

DC maturation was determined by measuring the upregulation of MHC class II molecules and “de novo” expression of CD83 using the fluorescence-activated cell sorter (FACS) analysis.

### Induction of CIA

Arthritis was induced in mice between 10 and 12 weeks of age. Bovine type II collagen was dissolved in 0.05 M acetic acid to a concentration of 2 mg/ml and emulsified in an equal volume of Freund's complete adjuvant (2 mg/ml of M. tuberculosis strain H37Ra; Difco laboratories).

Mice were immunized by intradermal injection of 100 ml of the emulsion at the base of the tail and were given an intraperitoneal booster injection of 100 ml of CII dissolved in phosphate-buffered saline on day 21. Clinical onset and progression of arthritis was macroscopically evaluated by two blinded observers and scored on a scale between 0 and 2 for each paw.

Treatment of Arthritis with TLR-4 Antagonist and TNFbp

To investigate the effects of TLR-4 inhibition on the development of arthritis compared to TNF blockade, mice with CIA were treated using 3 intraperitoneal injections of 400 μg/kg body weight TLR-4 antagonist or 3 mg/kg TNF binding protein (TNFbp, Enbrel; Amgen) once in two days started before the clinical onset of the disease.

For therapeutic treatment, mice received 4 daily injections of 2 mg/kg TLR-4 antagonist or 3 injections of 3 mg/kg TNFbp once every two days after a macroscopic inflammation score of 0.5-1 was reached on a scale up to 8. Development of arthritis was evaluated as described in the upper section.

BALB/c IL-1Ra^{-/-} mice received 400 μg/kg TLR-4 antagonist (saline as control) or 3 mg/kg TNFbp (3 mg/kg bovine serum albumin as control) three times a week during 2 weeks started after the spontaneous onset of arthritis.

### Histology

For histological assessment of arthritis, total knee joints were isolated on day 6 of the prophylactic and on day 4 of the therapeutic treatment and fixed during 4 days in 4% formaldehyde, then decalcified in 5% formic acid and embedded in paraffin.

Tissue sections of 7 μm were stained using the Haematoxylin & Eosin staining to study the inflammatory cell influx and chondrocyte death or using the Safranin O staining to determine PG-depletion and cartilage and bone destruction. Each parameter was scored on a scale from 0 to 3 by two observers in a blinded manner.

### Immunohistochemistry

Local expression of IL-1b was evaluated on paraffin sections of the knee joints. Sections were deparaffinized in xylol and rehydrated in serial dilutions of ethanol. Endogenous peroxidase was blocked using 1% hydrogen peroxide for 15 minutes.

Tissue sections were incubated with 7.5 mg/ml rabbit anti-mouse IL-1b antibodies or rabbit normal IgG (Santa Cruz) for 1 hour, followed by incubation with biotinylated swine anti-rabbit antibodies and peroxidase labeled streptavidin.

Color was developed with diaminobenzidine and tissues were counterstained with Haematoxylin. Il-1b expression was scored on articular chondrocytes (0-2) and synovial tissue around patella, tibia and femur (each scored between 0 and 2, then averaged to obtain overall expression in synovium).

### Measurement of Anti-Collagen Antibodies

Concentrations of anti-mouse CII IgG1 and IgG2a antibodies were determined using ELISA. Briefly, 96-wells plates were coated with 0.1 μg of mouse type II collagen (Chondrex). Non-specific binding sites were blocked by a 5% solution of milk powder. Serial dilutions of mice sera were added followed by incubation with isotype-specific goat-anti mouse antibodies (peroxidase labeled) and 5-aminosalicylic acid as substrate. Absorbance was measured at 450 nm.

### Statistical Analysis

Group measures are expressed as mean±S.E.M.

Statistical significance was assessed using non-paired two-tailed student t-test performed on GraphPad Prism 4.0 software (GraphPad software Inc., San Diego, Calif.). P values of 0.05 or less were considered significant

### Results and Discussion

Lipopolysaccharide Derived from Bartonella quintana is a Specific TLR-4 Antagonist

The TLR-4 antagonist used in this example was LPS derived from the cell membrane of the Gram-negative bacterium Bartonella quintana which was double purified using the phenol/water extraction method.

To determine the effect of B. quintana derived LPS on TLR-4 signaling, it was tested whether it could antagonize the proinflammatory properties of a known TLR-4 ligand, E. coli LPS.

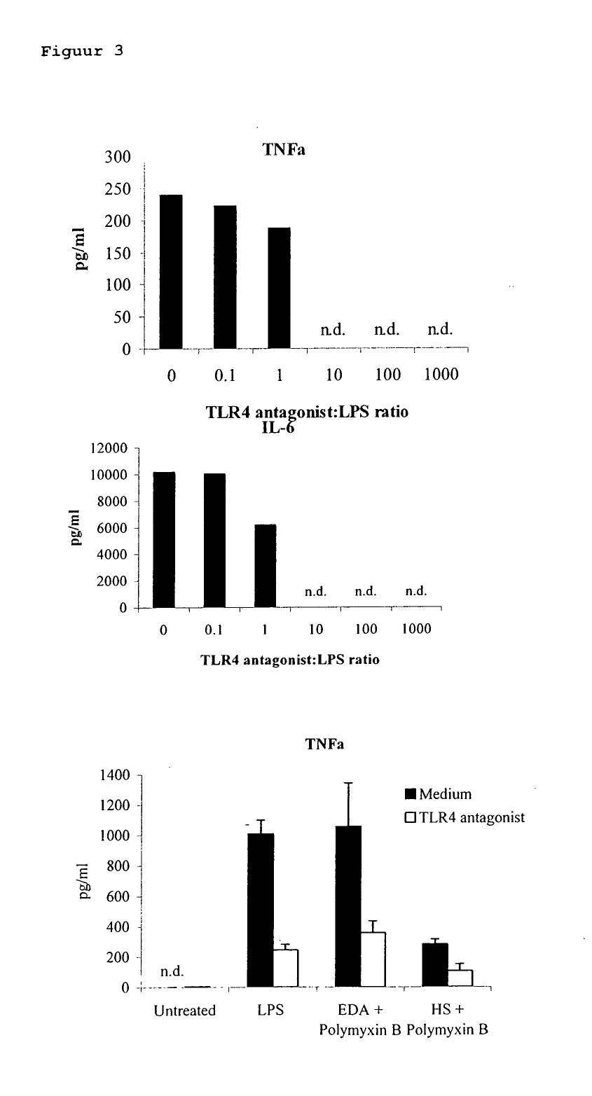

Human PBMCs were preincubated with various concentrations of B. quintana LPS and then exposed to 10 ng/ml purified E. coli LPS. Preincubation of PBMCs with B. quintana LPS resulted in an inhibition of TNFa and IL-6 production induced by E. coli LPS. A w/w ratio of 10:1 TLR-4 antagonist to LPS was sufficient to completely block the proinflammatory signals of E. coli LPS (FIGS. 3a and 3b). The same antagonistic activity was observed in mouse peritoneal macrophages upon stimulation with E. coli LPS (data not shown).

To confirm that B. quintana LPS is a specific TLR-4 antagonist and that it does not inhibit other sources of NF-kappaB activation, the effect were examined of B. quintana LPS on cytokine production induced by IL-1, TNFa and the synthetic TLR2 and TLR3 ligands Pam3Cys and Poly I:C, respectively.

As expected, pretreatment with the TLR-4 antagonist did not suppress the production of IL-6 and KC by peritoneal macrophages after exposure to IL-1, TNF, Pam3Cys and Poly I:C (data not shown).

Thus, B. quintana LPS acts as a specific TLR-4 antagonist and can inhibit the ability of E. coli LPS to signal through TLR-4, but it cannot inhibit cytokine release induced by other TLR ligands or proinflammatory mediators such as IL-1 and TNF.

### Characterization of the TLR-4 Antagonist

To further characterize the TLR-4 antagonist, the effects were determined of B. quintana LPS on cytokine production by mouse peritoneal macrophages and human peripheral blood mononuclear cells.

The TLR-4 antagonist did not by itself induce the production of proinflammatory cytokines such as IL-1, IL-6 and TNFalpha, nor did it induce the production of anti-inflammatory cytokines like IL-4, IL-10 and TGFbeta. The only inflammatory mediator induced by B. quintana LPS was the chemokine KC, which was produced at the concentration of 343 pg/ml after stimulation with 1 mg/ml B. quintana LPS.

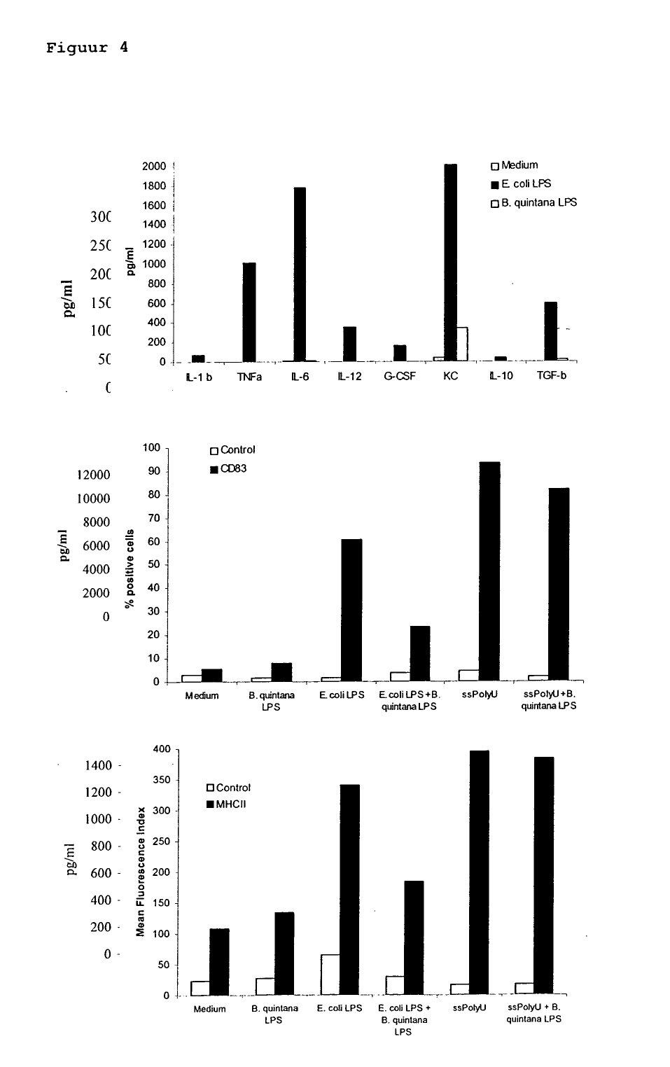

In comparison, stimulation of mouse peritoneal macrophages with only 10 ng/ml of purified E. coli LPS resulted in the production of as much as 2000 pg/ml KC (FIG. 4a).

In addition, B. quintana LPS, in contrast to E. coli LPS, did not induce the maturation of human monocyte-derived dendritic cells (DCs) in terms of induction of CD83 expression and MHC class II upregulation.

More importantly, B. quintana LPS had no inhibitory effect on DC maturation that was induced by other mechanisms than TLR-4 activation e.g. via stimulation of TLR7 (FIGS. 4b and 4c).

TLR-4 Antagonist Derived from Bartonella quintana Inhibits Proinflammatory Cytokine Production by Endogenous TLR-4 Ligands

There is growing evidence that extra-cellular matrix components, which are generated by tissue damage during chronic inflammation, can activate TLR-4. Therefore, the ability was examined of the TLR-4 antagonist to block the inflammatory signal induced by some of these endogenous TLR-4 ligands, i.e. the extra domain A (EDA) of fibronectin and heparan sulphate (HS).

Mouse peritoneal macrophages were preincubated with the TLR-4 antagonist and then stimulated with LPS, EDA or HS. As we expected, stimulation of macrophages with EDA or HS, which were premixed with excessive amounts of Polymyxin B to inhibit the putative LPS contaminant, resulted in the production of IL-1b and TNFalpha.

Preincubation of cells with the TLR-4 antagonist clearly inhibited the production of these cytokines by LPS and the endogenous TLR-4 ligands (FIG. 3c).

### Prophylactic Intervention of CIA Using TLR-4 Antagonist Inhibits Disease Progression

To investigate the role of TLR-4 in the development of arthritis, TLR-4 was inhibited in an experimental model of arthritis.

DBA-1 mice with collagen-induced arthritis were treated using three intraperitoneal injections of 400 mg/kg TLR-4 antagonist, 3 mg/kg TNFbp (Enbrel) or saline once every two days before clinical manifestation of the disease.

Macroscopic evaluation of the paws showed that TNFbp was better in suppressing the disease incidence (40% reduction) than TLR-4 antagonist (18% reduction); however, once arthritis has developed, both treatments could significantly suppress the macroscopic arthritis score to a comparable level at the endpoint of the experiment (d6).

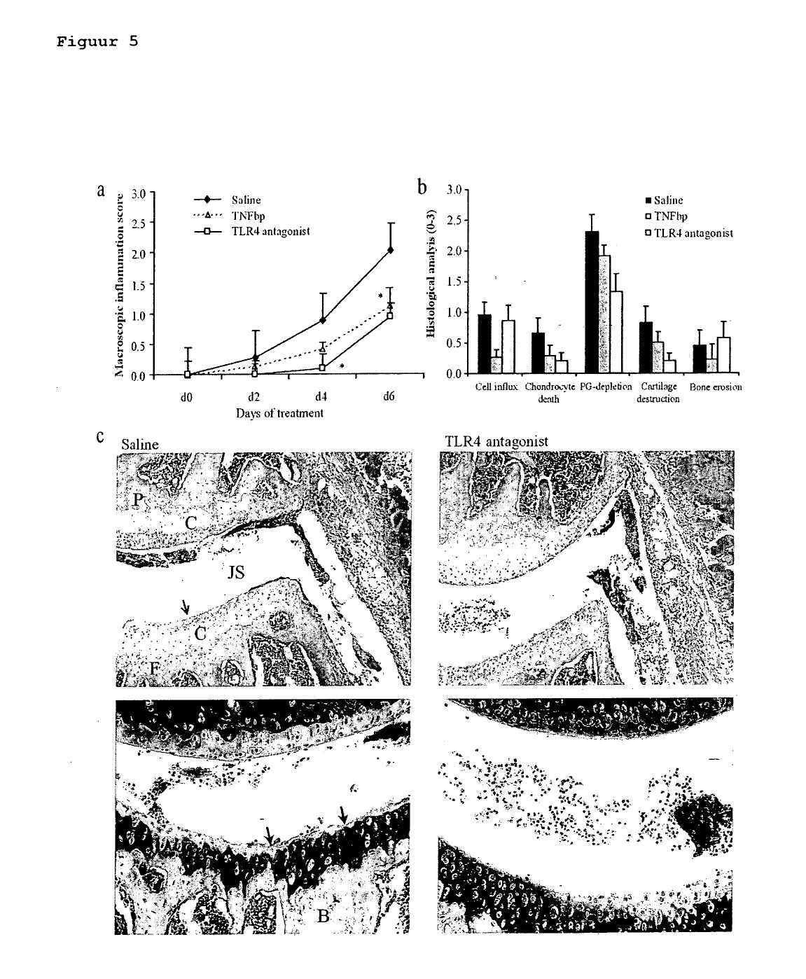

Interestingly, suppression of macroscopic inflammation score by TLR-4 antagonist was more pronounced than by TNFbp and was reached at an earlier time point (d4, FIG. 5c).

Subsequently, the effect was investigated of prophylactic TLR-4 blocking, relative to TNFbp, on the inflammatory cell influx and various hallmarks of cartilage and bone damage.

Analysis of the paraffin sections of the knee joints revealed that TNFbp inhibited the inflammatory cell influx into the joints, although TLR-4 antagonist did not.

Importantly, specific TLR-4 inhibition significantly suppressed proteoglycan depletion from the cartilage matrix, the earliest sign of cartilage damage in experimental arthritis.

Furthermore, destruction of the cartilage matrix was markedly reduced in mice treated with TLR-4 antagonist (P=0.014, both). None of these protective effects on cartilage were observed at significant levels in anti-TNF treated mice. The microscopic score of chondrocyte death was decreased by both treatments; however, it did not reach the significance level. The severity of bone erosion was not affected by the treatments in this setting (FIG. 5b).

Representative images of histological analysis demonstrating the effects of anti-TLR-4 treatment before the onset of CIA are shown in FIG. 5c.

### Therapeutic Treatment of CIA Using TLR-4 Antagonist Strongly Suppresses Joint Pathology in Ongoing Disease

Since inhibition of TLR-4 resulted in a moderate suppression of arthritis, it was investigated whether TLR-4 blocking could ameliorate the established disease in mice.

To ensure that TLR-4 activation by potential endogenous ligands in arthritic joints is completely blocked the dose of TLR-4 antagonist was enhanced.

Mice with collagen-induced arthritis received four daily injections of 2 mg/kg TLR-4 antagonist, three injections of 3 mg/kg TNFbp once every two days or saline intraperitoneally after a macroscopic inflammation score of 0.5-1 was reached on a scale between 0 and 8 for each mouse.

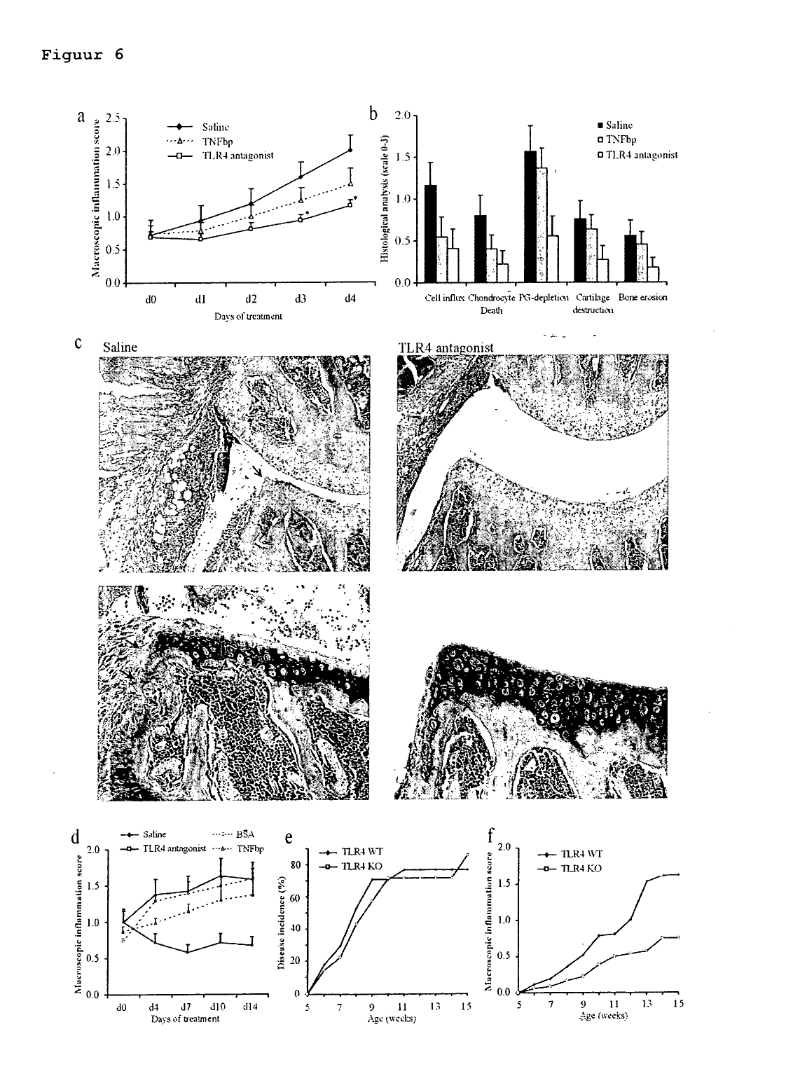

As shown in FIG. 6a, therapeutic treatment of CIA using the TLR-4 antagonist resulted in an approximately 50% suppression of the clinical score of arthritis, although TNFbp did not significantly suppress the disease progression in this setting.

Histological examination of the knee joints revealed that treatment with TLR-4 antagonist strongly prevented the PG-deletion and destruction of cartilage matrix. Chondrocyte death and infiltration of inflammatory cells into the joint space was also dramatically inhibited (P<0.05 for all parameters).

Furthermore, another characteristic hallmark of CIA, bone erosion, was moderately blocked by inhibition of TLR-4 signaling (P=0.067, FIG. 6b).

Among these pathological characteristics, anti-TNF treatment could only inhibit influx of inflammatory cells, underlining a more favorable effect of anti-TLR-4 treatment than anti-TNF based intervention in terms of protection against cartilage and bone damage. FIG. 6c shows representative images of the knee joints of mice with CIA treated with TLR-4 antagonist in comparison with saline.

### Anti-TLR-4 Treatment Reduces Local Production of IL-1b in the Joint

IL-1 is considered the main mediator of cartilage PG-depletion and destruction and bone erosion during CIA. Immunohistochemical staining of IL-1b showed that inhibition of TLR-4 before as well as after the onset of CIA resulted in a lower expression of IL-1b protein in the joints.

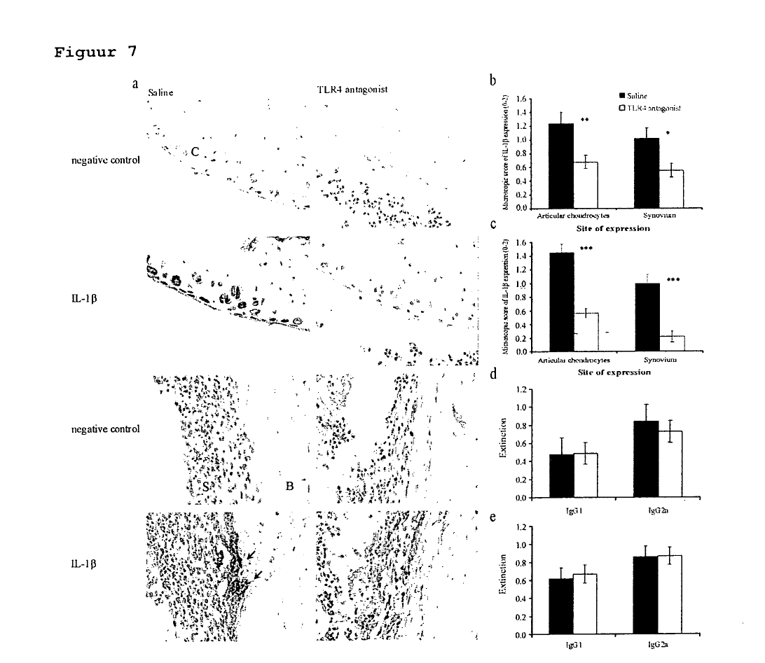

IL-1b expression was reduced in chondrocytes of articular cartilage and also in synovial tissue surrounding patellar, tibial and femoral surfaces of the knee joint (FIGS. 7a to 7c). In control mice, IL-1 was highly expressed in synovium, especially at the sites of bone erosion.

### Progression of Ongoing Arthritis in IL-1Ra^{-/-} Mice is Blocked by TLR-4 Inhibition or TLR-4 Gene Deficiency

Spontaneous development of arthritis in IL-1Ra^{-/-} mice reflects an IL-1 mediated autoimmune process progressing with age. To confirm the relevance of TLR-4 activation in driving IL-1 mediated joint pathology during the chronic phase of arthritis, IL-1Ra^{-/-} mice with an ongoing arthritis (mean macroscopic score of 1) were treated using intraperitoneal injection of 400 mg/kg TLR-4 antagonist or 3 mg/kg TNFbp three times a week during a period of two weeks.

In line with the findings in CIA, severity of joint inflammation was clearly reduced in anti-TLR-4 treated mice compared to the saline treated control group in which arthritis became more aggravated in time. In contrast, TNFbp was not capable of significant blocking of disease progression as compared to BSA treated control animals (FIG. 6d).

The role of TLR-4 was confirmed by strong suppression of clinical inflammation score in IL-1Ra^{-/-} mice lacking the TLR-4 gene as compared to IL-1Ra^{-/-} TLR-4-wild type (WT) littermates, which developed aggressive arthritis mainly in hind paws.

Disease severity was reduced by 54% in TLR-4^{-/-} mice, especially during the more-progressed chronic phase of arthritis (13-15 weeks of age), even though the disease incidence was comparable in both groups (FIGS. 6e and 6f).

### Serum Cytokine and Anti-Collagen Type II Antibody Levels

Treatment of mice with the TLR-4 antagonist before the onset of CIA did not result in any difference in serum concentrations of cytokines and chemokines.

In contrast, inhibition of TLR-4 after the arthritis onset led to a reduction in serum levels of IL-6 and KC, which corresponded well with reduction of inflammatory cell influx into the joint (data not shown).

IL-1b and TNFalpha concentrations were not different in the two groups. Importantly, serum levels of anti-mouse collagen type II antibodies in mice treated with TLR-4 antagonist in both prophylactic and therapeutic settings did not differ from that of control mice (FIGS. 7d and 7e).

This finding confirms that treatment with TLR-4 antagonist did not interact with the development of autoimmune responses driving the initiation and expression of CIA.

Suppression of CIA by B. quintana LPS is Mediated by TLR-4 Antagonism; not by Induction of Corticosteroids or Anti-Inflammatory Cytokines

To exclude that the inhibitory effect of the TLR-4 antagonist on progression of arthritis is mediated through the induction of anti-inflammatory cytokines or corticosteroids, naive male DBA-1 mice were injected with 50 mg of the antagonist, 10 mg E. coli LPS or an equal volume of saline intraperitoneally.

Serum was isolated at 90 minutes, 4 hours and 24 hours after injection, and corticosterone and cytokine concentrations were measured in serum. As shown in FIGS. 8a to 8d, systemic injection of E. coli LPS resulted in the production of high amounts of TNFalpha and IL-6 and low concentrations of IL-1b. In addition, injection of E. coli LPS resulted in the production of the anti-inflammatory cytokine IL-10.

In contrast to E. coli LPS, B. quintana LPS did not induce the production of any of the cytokines mentioned above, indicating that the binding of B. quintana-derived LPS to the TLR-4 receptor complex does not lead to the common NFkappaB activation.

These cytokines were not detectable in serum 24 hours after injection except IL-10 (<20 pg/ml). Furthermore, no IL-4 was detectable in serum of any of the groups at various time points. These data were in agreement with cytokine measurements in vitro showing that B. quintana LPS does not induce the production of these cytokines.

In order to rule out corticosteroid induction as a mechanism for the inhibition of joint inflammation and cartilage damage by B. quintana LPS, corticosterone concentrations were determined in serum.

As FIG. 8e shows, injection of E. coli LPS led to the production of high levels of corticosterone within 90 minutes, whereas animals treated with B. quintana LPS had corticosterone levels comparable to that of saline-treated animals.

Since the B. quintana LPS is capable of inducing the mouse chemokine KC by macrophages, the stimulatory effect of B. quintana LPS injection on the chemotaxis of monocytes and neutrophils into the peritoneal cavity was examined to exclude that the reduction of inflammation in arthritic joints has been a result of this chemotaxis.

Twenty-four hours after intraperitoneal injection of the B. quintana LPS, E. coli LPS or saline, peritoneal cells were collected and counted, then stained using the May-Grunwald-Giemsa staining method. The total number of cells isolated from the peritoneal cavity was comparable in all groups (1.30×10⁵ cells/ml in control mice versus 1.38×10⁵ and 1.33×10⁵ cells/ml in B. quintana LPS and E. coli LPS injected mice, respectively).

Furthermore, systemic injection of B. quintana LPS had no consequences for the types of cells available in the peritoneal cavity: Peritoneal cell populations in all groups consisted mainly of macrophages (>99.8%); however, low percentages of mast cell-like cells (0.15, 0.11 and 0.14% in control, B. quintana LPS injected and E. coli LPS injected mice, respectively) and sporadically eosinophils (0.02%) were also observed.

Moreover, study of the peritoneal cell population revealed that no PMNs were recruited to the peritoneal cavity. This confirms that suppression of inflammation and tissue damage in arthritic joints is not mediated by strong attraction of inflammatory cells to the site of injection. Altogether, these data provide evidence of the inhibitory effect of TLR-4 blocking on joint inflammation and cartilage and bone destruction in experimental arthritis.

### CONCLUSION

Degeneration of extracellular matrix of cartilage leads to the production of molecules capable of activating innate and adaptive immune responses via Toll-like receptor 4 (TLR-4). In the present example, it was demonstrated that treatment of collagen-induced arthritis using a naturally occurring TLR-4 antagonist, a highly purified lipopolysaccharide from Bartonella quintana, substantially suppresses both clinical and histological characteristics of arthritis, without influencing the adaptive anti-type II collagen immunity crucial for this model.

Anti-TLR-4 treatment was superior to anti-TNF therapy in protecting chondrocytes and cartilage from damage, and strongly reduced IL-1b expression in the joint.

Furthermore, treatment with TLR-4 antagonist, but not with anti-TNF, inhibited the IL-1-mediated autoimmune arthritis in IL-1 receptor antagonist (IL-1Ra) deficient mice.

In accordance, IL-1Ra/TLR-4 double deficient mice showed markedly reduced severity of arthritis.

This demonstrates the involvement of TLR-4 signaling in the pathogenesis of RA and TLR-4 is a novel target in the treatment of RA.

1. Use of Bartonella quintana lipopolysaccharide or a functional equivalent fragment thereof for the preparation of a medicament for the treatment of an autoimmune disease, an autoinflammatory disease, or an infectious disease in a mammal.

2. Use according to claim 1, wherein the mammal is a human mammal.

3. Use according to claim 2, wherein the treatment comprises inhibition of the toll-like receptor signaling.

4. Use according to claim 1, wherein the treatment comprises inhibition of the toll-like receptor 4 (TLR-4)

5. Use according to claim 1, wherein the autoimmune or inflammatory disease is selected from the group consisting of rheumatoid arthritis, Crohn's disease, bacterial, viral and fungal infections, bacterial, viral and fungal infections resulting in secondary side-effects, SIRS, sepsis, atherosclerosis, type I diabetes mellitus, Wegener's granulomatosis, ulcerative colitis, systemic lupus erythemotosis (SLE), psoriasis, and multiple sclerosis.

6. Use according to claim 5, wherein the autoimmune disease is rheumatoid arthritis.

7. Bartonella quintana lipopolysaccharide or a functional equivalent fragment thereof for use as a medicament.

8. Medicament comprising Bartonella quintana lipopolysaccharide or a functional equivalent fragment thereof and one or more pharmaceutically acceptable excipients and/or carriers.

9. Method for treating an autoimmune or inflammatory disease comprising administering an therapeutic amount of Bartonella quintana lipopolysaccharide or a functional equivalent fragment thereof to a mammal suffering from an autoimmune, an autoinflammatory, or an infectious disease.

10. Method according to claim 9, wherein the mammal is a human mammal.

11. Use of a Toll-like 4 receptor (TLR-4) antagonist for the preparation of a medicament for the treatment of rheumatoid arthritis.

12. Use according to claim 11, wherein the Toll-like 4 receptor (TLR-4) antagonist is Bartonella quintana lipopolysaccharide (LPS) or a functional equivalent thereof.
